(19) United States
(12) Patent Application Publication
(10) Pub. No.: US 2007/0104685 A1
(43) Pub. Date: May 10, 2007

(54) Synthetic gene encoding human carcinoembryonic antigen and uses thereof

(76) Inventors: Nicola La Monica, Pomezia (IT); Armin Lahm, Peomezia (IT); Carmela Mennuni, Promezia (IT); Rocco Savino, Promezia (IT)

Correspondence Address: MERCK AND CO., INC, P O BOX 2000, RAHWAY, NJ, 07065-0907, US

(21) Appl. No.: 10/555,744

(22) PCT Filed: May 3, 2004
(86) PCT No.: PCT/EP04/04802

§ 371 Date: Nov. 20, 2006

Related U.S. Application Data
(60) Provisional application No. 60/467,971, filed on May 5, 2003.
(60) Provisional application No. 60/543,612, filed on Feb. 11, 2004.

(51) Int. Cl. A61K 48/00; C12Q 1/68; C07H 21/04; C12P 21/06; C12N 15/861; C07K 14/82

(52) U.S. Cl. 424/93.2; 435/6; 435/69.1; 435/320.1; 435/456; 435/325; 530/350; 536/23.5

(57) ABSTRACT

Synthetic polynucleotides encoding human carcinoembryonic antigen (CEA) are provided, the synthetic polynucleotides being codon-optimized for expression in a human cellular environment. The gene encoding CEA is commonly associated with the development of human carcinomas. The present invention provides compositions and methods to elicit or enhance immunity to the protein product expressed by the CEA tumor-associated antigen, wherein aberrant CEA expression is associated with a carcinoma or its development. This invention specifically provides adenoviral vector and plasmid constructs carrying codon-optimed human CEA and discloses their use in vaccines and pharmaceutical compositions for preventing and treating cancer.

### FIELD OF THE INVENTION

The present invention relates generally to the therapy of cancer. More specifically, the present invention relates to synthetic polynucleotides encoding the human tumor associated polypeptide carcinoembryonic antigen, herein designated hCEAopt, wherein the polynucleotides are codon-optimized for expression in a human cellular environment. The present invention also provides recombinant vectors and hosts comprising said synthetic polynucleotides. This invention also relates to adenoviral vector and plasmid constructs carrying hCEAopt and to their use in vaccines and pharmaceutical compositions for preventing and treating cancer.

### BACKGROUND OF THE INVENTION

The immunoglobulin superfamily (IgSF) consists of numerous genes that code for proteins with diverse functions, one of which is intercellular adhesion. IgSF proteins contain at least one Ig-related domain that is important for maintaining proper intermolecular binding interactions. Because such interactions are necessary to the diverse biological functions of the IgSF members, disruption or aberrant expression of many IgSF adhesion molecules has been correlated with many human diseases.

The carcinoembryonic antigen (CEA) belongs to a subfamily of the Ig superfamily consisting of cell surface glycoproteins. Members of the CEA subfamily are known as CEA-related cell adhesion molecules (CEACAMs). In recent scientific literature, the CEA gene has been renamed CEACAM5, although the nomenclature for the protein remains CEA. Functionally, CEACAMs have been shown to act as both homotypic and heterotypic intercellular adhesion molecules (Benchimol et al., Cell 57: 327-334 (1989)). In addition to cell adhesion, CEA inhibits cell death resulting from detachment of cells from the extracellular matrix and can contribute to cellular transformation associated with certain proto-oncogenes such as Bcl2 and C-Myc (see Berinstein, J. Clin. Oncol. 20(8): 2197-2207 (2002)).

Normal expression of CEA has been detected during fetal development and in adult colonic mucosa. CEA overexpression was first detected in human colon tumors over thirty years ago (Gold and Freedman, J. Exp. Med. 121:439-462 (1965)) and has since been found in nearly all colorectal tumors. Additionally, CEA overexpression is detectable in a high percentage of adenocarcinomas of the pancreas, breast and lung. Because of the prevalence of CEA expression in these tumor types, CEA is widely used clinically in the management and prognosis of these cancers.

Sequences coding for human CEA have been cloned and characterized (U.S. Pat. No. 5,274,087; U.S. Pat. No. 5,571,710; and U.S. Pat. No. 5,843,761. See also Beauchernin et al., Mol. Cell. Biol. 7:3221-3230 (1987); Zimmerman et al., Proc. Natl. Acad. Sci. USA 84:920-924 (1987); Thompson et al. Proc. Natl. Acad. Sci. USA 84(9):2965-69 (1987)).

The correlation between CEA expression and metastatic growth has led to its identification as a target for molecular and immunological intervention for colorectal cancer treatment. One therapeutic approach targeting CEA is the use of anti-CEA antibodies (see Chester et al., Cancer Chemother. Pharmacol. 46 (Suppl): S8-S12 (2000)), while another is to activate the immune system to attack CEA-expressing tumors using CEA-based vaccines (for review, see Berinstein, supra).

The development and commercialization of many vaccines have been hindered by difficulties associated with obtaining high expression levels of exogenous genes in successfully transformed host organisms. Therefore, despite the identification of the wild-type nucleotide sequences encoding CEA proteins described above, it would be highly desirable to develop a readily renewable source of human CEA protein that utilizes CEA-encoding nucleotide sequences that are optimized for expression in the intended host cell, said source allowing for the development of a cancer vaccine which is efficacious and not hindered by self-tolerance.

### SUMMARY OF THE INVENTION

The present invention relates to compositions and methods to elicit or enhance immunity to the protein products expressed by CEA genes, which have been associated with numerous adenocarcinomas, including colorectal carcinomas. Specifically, the present invention provides polynucleotides encoding human CEA protein, wherein said polynucleotides are codon-optimized for high level expression in a human cell. The present invention further provides adenoviral and plasmid-based vectors comprising the synthetic polynucleotides and discloses use of said vectors in immunogenic compositions and vaccines for the prevention and/or treatment of CEA-associated cancer.

The present invention also relates to synthetic nucleic acid molecules (polynucleotides) comprising a sequence of nucleotides that encode human carcinoembryonic antigen (hereinafter hCEA) as set forth in SEQ ID NO:2, wherein the synthetic nucleic acid molecules are codon-optimized for high-level expression in a human cell (hereinafter hCEAopt). The nucleic acid molecules disclosed herein may be transfected into a host cell of choice wherein the recombinant host cell provides a source for substantial levels of an expressed functional hCEA protein (SEQ ID NO:2).

The present invention further relates to a synthetic nucleic acid molecule which encodes mRNA that expresses a human CEA protein; this DNA molecule comprising the nucleotide sequence disclosed herein as SEQ ID NO:1. A preferred aspect of this portion of the present invention is disclosed in FIG. 1, which shows a DNA molecule (SEQ ID NO:1) that encodes a hCEA protein (SEQ ID NO:2 or SEQ ID NO:16). The preferred nucleic acid molecule of the present invention is codon-optimized for high-level expression in a human cell.

Another preferred DNA molecule of the present invention comprises a sequence of nucleotides that encodes a human CEA that is deleted of its C-terminal anchoring domain (AD), which is located from about amino acid 679 to about amino acid 702 of the human full-length CEA (SEQ ID NO:2), wherein said sequence of nucleotides is codon-optimized for high level expression in a human cell. An exemplary DNA molecule encoding a CEA variant that is truncated of its anchoring domain is set forth in SEQ ID NO:15 (shown in FIG. 10A). The corresponding amino acid sequence of hCEA-ΔAD is set forth in SEQ ID NO:16 (shown in FIG. 10B).

The present invention also relates to recombinant vectors and recombinant host cells, both prokaryotic and eukaryotic, which contain the nucleic acid molecules disclosed throughout this specification.

The present invention further relates to a process for expressing a codon-optimized human CEA protein in a recombinant host cell, comprising: (a) introducing a vector comprising a nucleic acid molecule as set forth in SEQ ID NO:1 or SEQ ID NO:15 into a suitable host cell; and, (b) culturing the host cell under conditions which allow expression of said codon-optimized human protein.

Another aspect of this invention is a method of preventing or treating cancer comprising adnministering to a mammal a vaccine vector comprising a synthetic nucleic acid molecule, the synthetic nucleic acid molecule comprising a sequence of nucleotides that encodes a human carcinoembryonic antigen (hCEA) protein as set forth in SEQ ID NO:2 or SEQ ID NO:16, wherein the synthetic nucleic acid molecule is codon-optimized for high level expression in a human cell.

The present invention further relates to an adenovirus vaccine vector comprising an adenoviral genome with a deletion in the E1 region, and an insert in the E1 region, wherein the insert comprises an expression cassette comprising: (a) a codon-optimized polynucleotide encoding a human CEA protein; and (b) a promoter operably linked to the polynucleotide.

The present invention also relates to a vaccine plasmid comprising a plasmid portion and an expression cassette portion, the expression cassette portion comprising: (a) a synthetic polynucleotide encoding a human CEA protein, wherein the synthetic polynucleotide is codon-optimized for optimal expression in a human cell; and (b) a promoter operably linked to the polynucleotide.

Another aspect of the present invention is a method of protecting or treating a mammal from cancer or treating a mammal suffering from CEA-associated cancer comprising: (a) introducing into the mammal a first vector comprising: i) a codon-optimized polynucleotide encoding a human CEA protein; and ii) a promoter operably linked to the polynucleotide; (b) allowing a predetermined amount of time to pass; and (c) introducing into the mammal a second vector comprising: i) a codon-optimized polynucleotide encoding a human CEA protein; and ii) a promoter operably linked to the polynucleotide.

As used throughout the specification and in the appended claims, the singular forms “a,” “an,” and “the” include the plural reference unless the context clearly dictates otherwise.

As used throughout the specification and appended claims, the following definitions and abbreviations apply:

The term “promoter” refers to a recognition site on a DNA strand to which the RNA polymerase binds. The promoter forms an initiation complex with RNA polymerase to initiate and drive transcriptional activity. The complex can be modified by activating sequences termed “enhancers” or inhibiting sequences termed “silencers”.

The term “cassette” refers to the sequence of the present invention that contains the nucleic acid sequence which is to be expressed. The cassette is similar in concept to a cassette tape; each cassette has its own sequence. Thus by interchanging the cassette, the vector will express a different sequence. Because of the restriction sites at the 5′ and 3′ ends, the cassette can be easily inserted, removed or replaced with another cassette.

The term “vector” refers to some means by which DNA fragments can be introduced into a host organism or host tissue. There are various types of vectors including plasmid, virus (including adenovirus), bacteriophages and cosmids.

The term “first generation,” as used in reference to adenoviral vectors, describes said adenoviral vectors that are replication-defective. First generation adenovirus vectors typically have a deleted or inactivated E1 gene region, and preferably have a deleted or inactivated E3 gene region.

The designation “pV1J/hCEAopt” refers to a plasmid construct disclosed herein comprising the human CMV immediate-early (IE) promoter with intron A, a full-length codon-optimized human CEA gene, bovine growth hormone-derived polyadenylation and transcriptional termination sequences, and a minimal pUC backbone (see EXAMPLE 2). The designation “pV1J/hCEA” refers to a construct as described above, except the construct comprises a wild-type human CEA gene instead of a codon-optimized human CEA gene.

The designations “MRKAd5/hCEAopt” and “MRKd5/hCEA” refer to two constructs, disclosed herein, which comprise an Ad5 adenoviral genome deleted of the E1 and E3 regions. In the “MRKAd5/hCEAopt” construct, the E1 region is replaced by a codon-optimized human CEA gene in an E1 parallel orientation under the control of a human CMV promoter without intron A, followed by a bovine growth hormone polyadenylation signal. The “MRKAd5/hCEA” construct is essentially as described above, except the E1 region of the Ad5 genome is replaced with a wild-type human CEA sequence (see EXAMPLE 2).

The term “effective amount” means sufficient vaccine composition is introduced to produce the adequate levels of the polypeptide, so that an immune response results. One skilled in the art recognizes that this level may vary.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

“hCEA” and “hCEAopt” refer to a human carcinoembryonic antigen and a human codon-optimized carcinoembryonic antigen, respectively.

The term “hCEA-ΔAD” refers to a variant of human CEA that is deleted of its C-terminal anchoring domain (AD), which is located from about amino acid 679 to about amino acid 702 of the human full-length CEA (SEQ ID NO:2). Nucleotide sequences encoding hCEA-ΔAD of the present invention. are codon-optimized for high-level expression in a human cellular environment (designated herein hCEAopt-ΔAD”). An exemplary DNA molecule encoding a CEA variant that is truncated of its anchoring domain is set forth in SEQ ID NO:15 (shown in FIG. 10A). The corresponding amino acid sequence of hCEA-ΔAD is set forth in SEQ ID NO:16 (shown in FIG. 10B). Nucleotides encoding hCEA-ΔAD are useful for the development of a cancer vaccine for treatment and/or prophylaxis of cancer.

The term “mammalian” refers to any mammal, including a human being.

The abbreviation “Ag” refers to an antigen.

The abbreviations “Ab” and “mAb” refer to an antibody and a monoclonal antibody, respectively.

The abbreviation “ORF” refers to the open reading frame of a gene.

### BRIEF DESCRIPTION OF THE DRAWINGS

FIG. 1 shows the nucleotide sequence of wild-type human CEA cDNA (SEQ ID NO:3) and of the codon optimized clone (hCEAopt, SEQ ID NO:1). The deduced amino acid sequence is shown on top (SEQ ID NO:2). The substituted nucleotides of the synthetic codon optimized cDNA are shown below the hCEA cDNA sequence. See EXAMPLE 2.

FIG. 2 shows the expression of hCEA in injected mice. Groups of 10 C57BL/6 mice were injected in the quadriceps muscle either with various doses of MRKAd5-hCEA and MRKAd5-hCEAopt (Panel A) or with 25 or 50 micrograms of plasmids pV1J/hCEA and pV1J/hCEAopt (Panel B). Blood samples were collected 3 days postinjection and CEA levels were measured. Filled triangles represent CEA measurement of individual mice. Geometric mean values are also shown (filled circle).

FIG. 3 shows that codon optimization increases the immune response to human CEA. Groups of 8 C57BL/6 mice were injected via the quadriceps muscle either with various doses of MRKAd5-hCEA and MRKAd5-hCEAopt. Virus injections were carried out at 0 and 21 days. Panel A. At two weeks post boosting injection, the number of CD8+ IFNγ secreting T cells specific for hCEA was determined by ELISPOT assay on splenocytes from individual mice (filled triangles) using peptide 143 that covers aa 569-583 and includes a CD8+ epitope. Two different amounts of splenocytes (2.5×10⁵ and 5×10⁵) and two replicas of each tested amount of splenocytes. Average values were calculated by subtracting the background level determined in the absence of peptides (typically less than 10 SFC/10⁶ total splenocytes), and the results were expressed as the number of SFC/10⁶ total splenocytes. Values from individual mice are shown (filled triangles) as well as the geometric mean values (filled circle). Panel B. Anti-A antibody titers in sera from individual mice (filled triangles) were measured using 10 days post boost serum samples. Geometric mean titers (GMT) (filled circles) are also shown. Ad/hCEAopt is significantly different from Ad/hCEA.

FIG. 4. Comparison of different immunization regimens. Groups of C57BL/6 (A) or BALB/c (B) mice were immunized with different combinations of plasmid pV1J/hCEA (50 μg/dose electroinjected in the quadriceps muscle) and MRKAd5/hCEA (1×10⁹ pp/dose). The number of IFNγ-secreting T cells in splenocytes in each individual mouse was determined using a pool of peptides covering aa 497-703 (pool D) as described in materials and methods and in the legend to FIG. 3. Geometric mean values are also shown (filled circles). D/D and D/A are significantly different from Ad/Ad group in C57BL/6 mice. All three groups are significantly different in BALB/c mice.

FIG. 5 shows the results of mapping of T-cell responses to selected regions of the hCEA protein. Groups of C57BL/6 (Panel A) or BALB/c (Panel B) mice were immunized with 50 μg of plasmid pV1J/hCEA and boosted three weeks later with 1×10⁹ pp of Ad/hCEA. The number of IFNγ-secreting T cells in splenocytes in each individual mouse was determined two weeks post-boost using pool of peptides covering the entire protein as described in materials and methods and in the legend to FIG. 3. Geometric mean values are also shown (filled circles).

FIG. 6. Identification of immunoresponsive peptides of hCEA. Pooled splenocytes from 4 immunized C57BL/6 (Panel A) or BALB/c (Panel B) mice were assayed for IFNγ secretion against each indicated peptide by ELISPOT assay (see EXAMPLE 8).

FIG. 7 shows the sequence of epitope containing peptides for CEA in C57BL/6 mice (Panel A) and BALB/c mice (Panel B) (see EXAMPLE xx). Listed to the right are the percent of IFNγ producing CD8+ (CD4+) CD3+ cells.

FIG. 8 shows results from an IFNγ-ELISPOT assay of immunized CEA transgenic mice as described in EXAMPLE 9. Mice were immunized with four electroinjections of plasmid DNA one week apart plus one adenovirus injection. For each immunogen, data were obtained with pooled splenocytes of three injected mice. The CD8-specific response was measured using peptide 143.

FIG. 9 shows IFNγ-intracellular staining of immunized CEA.tg mice. Mice were immunized with 2 injections of 1×10¹⁰ vp of Adenovirus two weeks apart. Shown are the data obtained with pooled splenocytes of three injected mice. Listed to the right are the percent of CD8+ or CD4+ cells.

FIG. 10, Panel A, shows an exemplary codon-optimized DNA molecule encoding a CEA variant that is truncated of its anchoring domain as set forth in SEQ ID NO:15. The corresponding amino acid sequence of hCEA-ΔAD is shown in Panel B (SEQ ID NO:16).

### DETAILED DESCRIPTION OF THE INVENTION

Carcinoembryonic antigen (CEA) is commonly associated with the development of adenocarcinomas. The present invention relates to compositions and methods to elicit or enhance immunity to the protein product expressed by the CEA tumor-associated antigen, wherein aberrant CEA expression is associated with the carcinoma or its development. Association of aberrant CEA expression with a carcinoma does not require that the CEA protein be expressed in tumor tissue at all timepoints of its development, as abnormal CEA expression may be present at tumor initiation and not be detectable late into tumor progression or vice-versa.

To this end, synthetic DNA molecules encoding the human CEA protein are provided. The codons of the synthetic molecules are designed so as to use the codons preferred by the projected host cell, which in preferred embodiments is a human cell. The synthetic molecules may be used for the development of recombinant adenovirus or plasmid-based vaccines, which provide effective immunoprophylaxis against CEA-associated cancer through neutralizing antibody and cell-mediated immunity. The synthetic molecules may be used as an immunogenic composition. This invention provides polynucleotides which, when directly introduced into a vertebrate in vivo, including mammals such as primates and humans, induce the expression of encoded proteins within the animal.

The wild-type human CEA nucleotide sequence has been reported (See, e.g., U.S. Pat. No. 5,274,087; U.S. Pat. No. 5,571,710; and U.S. Pat. No. 5,843,761). The present invention provides synthetic DNA molecules encoding the human CEA protein. The synthetic molecules of the present invention comprise a sequence of nucleotides, wherein some of the nucleotides have been altered so as to use the codons preferred by a human cell, thus allowing for high-level expression of CEA in a human host cell. The synthetic molecules may be used as a source of CEA protein, which may be used in a cancer vaccine to provide effective immunoprophylaxis against CEA-associated carcinomas through neutralizing antibody and cell-mediated immunity.

A “triplet” codon of four possible nucleotide bases can exist in over 60 variant forms. Because these codons provide the message for only 20 different amino acids (as well as transcription initiation and termination), some amino acids can be coded for by more than one codon, a phenomenon known as codon redundancy. For reasons not completely understood, alternative codons are not uniformly present in the endogenous DNA of differing types of cells. Indeed, there appears to exist a variable natural hierarchy or “preference” for certain codons in certain types of cells. As one example, the amino acid leucine is specified by any of six DNA codons including CTA, CTC, CTG, CTT, TTA, and TTG. Exhaustive analysis of genome codon frequencies for microorganisms has revealed endogenous DNA of E. coli most commonly contains the CTG leucine-specifying codon, while the DNA of yeasts and slime molds most commonly includes a TTA leucine-specifying codon. In view of this hierarchy, it is generally believed that the likelihood of obtaining high levels of expression of a leucine-rich polypeptide by an E. coli host will depend to some extent on the frequency of codon use. For example, it is likely that a gene rich in TTA codons will be poorly expressed in E. coli, whereas a CTG rich gene will probably be highly expressed in this host. Similarly, a preferred codon for expression of a leucine-rich polypeptide in yeast host cells would be TTA.

The implications of codon preference phenomena on recombinant DNA techniques are manifest, and the phenomenon may serve to explain many prior failures to achieve high expression levels of exogenous genes in successfully transformed host organisms—a less “preferred” codon may be repeatedly present in the inserted gene and the host cell machinery for expression may not operate as efficiently. This phenomenon suggests that synthetic genes which have been designed to include a projected host cell's preferred codons provide an optimal form of foreign genetic material for practice of recombinant DNA techniques. Thus, one aspect of this invention is a human CEA gene that is codon-optimized for expression in a human cell. In a preferred embodiment of this invention, it has been found that the use of alternative codons encoding the same protein sequence may remove the constraints on expression of exogenous CEA protein in human cells.

In accordance with this invention, the human CEA gene sequence was converted to a polynucleotide sequence having an identical translated sequence but with alternative codon usage as described by Lathe, “Synthetic Oligonucleotide Probes Deduced from Amino Acid Sequence Data: Theoretical and Practical Considerations” J. Molec. Biol. 183:1-12 (1985), which is hereby incorporated by reference. The methodology generally consists of identifying codons in the wild-type sequence that are not commonly associated with highly expressed human genes and replacing them with optimal codons for high expression in human cells. The new gene sequence is then inspected for undesired sequences generated by these codon replacements (e.g., “ATTTA” sequences, inadvertent creation of intron splice recognition sites, unwanted restriction enzyme sites, etc.). Undesirable sequences are eliminated by substitution of the existing codons with different codons coding for the same amino acid. The synthetic gene segments are then tested for improved expression.

The methods described above were used to create synthetic gene sequences for human CEA, resulting in a gene comprising codons optimized for high level expression. While the above procedure provides a summary of our methodology for designing codon-optimized genes for use in cancer vaccines, it is understood by one skilled in the art that similar vaccine efficacy or increased expression of genes may be achieved by minor variations in the procedure or by minor variations in the sequence. One of skill in the art will also recognize that additional DNA molecules may be constructed that provide for high levels of CEA expression in human cells, wherein only a portion of the codons of the DNA molecules are codon-optimized.

Accordingly, the present invention relates to a synthetic polynucleotide comprising a sequence of nucleotides encoding a human CEA protein (SEQ ID NO:2), or a biologically active fragment or mutant form of a human CEA protein, including, but not limited to hCEA-ΔAD (SEQ ID NO:16), the polynucleotide sequence comprising codons optimized for expression in a human host. Said mutant forms of the CEA protein include, but are not limited to: conservative amino acid substitutions, amino-terminal truncations, carboxy-terminal truncations, deletions, or additions, collectively referred to herein as “variants”. Any such biologically active fragment and/or mutant will encode either a protein or protein fragment which at least substantially mimics the immunological properties of the CEA protein as set forth in SEQ ID NO:2. The synthetic polynucleotides of the present invention encode mRNA molecules that express a functional human CEA protein so as to be useful in the development of a therapeutic or prophylactic cancer vaccine.

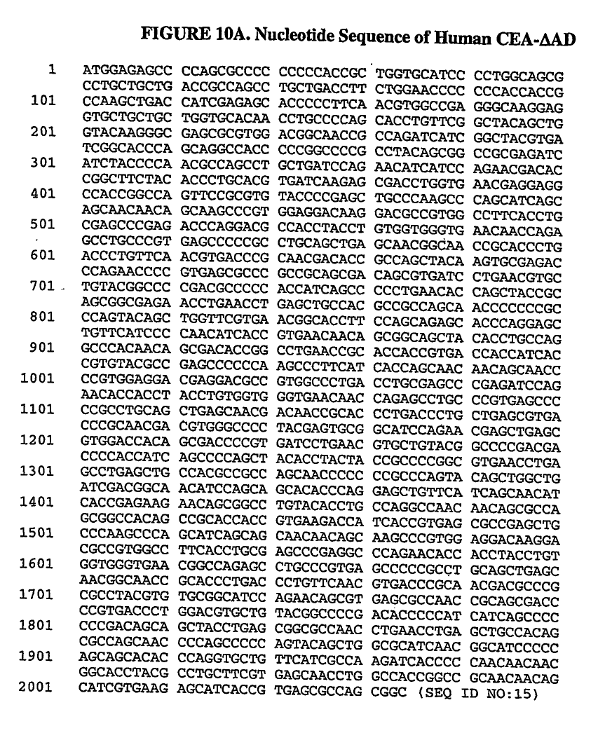

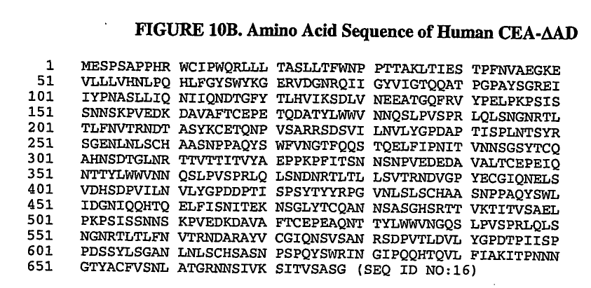

As stated above, the present invention relates to nucleotides encoding a human CEA protein (SEQ ID NO:2), or a biologically active fragment or mutant form thereof. To this end, the present invention provides nucleotides encoding hCEA-ΔAD (SEQ ID NO:16, FIG. 10B), which comprises a human CEA protein that is deleted of its C-terminal anchoring sequence. The nucleic acid molecules of the present invention encoding hCEA-ΔAD are codon-optimized for enhanced expression in human cells. An exemplary nucleic acid molecule encoding hCEA-ΔAD comprises a sequence of nucleotides as set forth in SEQ ID NO:15 (FIG. 10A).

The present invention relates to an synthetic nucleic acid molecule (polynucleotide) comprising a sequence of nucleotides which encodes mRNA that expresses a novel hCEA protein as set forth in SEQ ID NO:2, wherein the synthetic nucleic acid molecule is codon-optimized for high-level expression in a human host cell. The nucleic acid molecules of the present invention are substantially free from other nucleic acids.

The present invention also relates to recombinant vectors and recombinant host cells, both prokaryotic and eularyotic, which contain the nucleic acid molecules disclosed throughout this specification. The synthetic DNA molecules, associated vectors, and hosts of the present invention are useful for the development of a cancer vaccine.

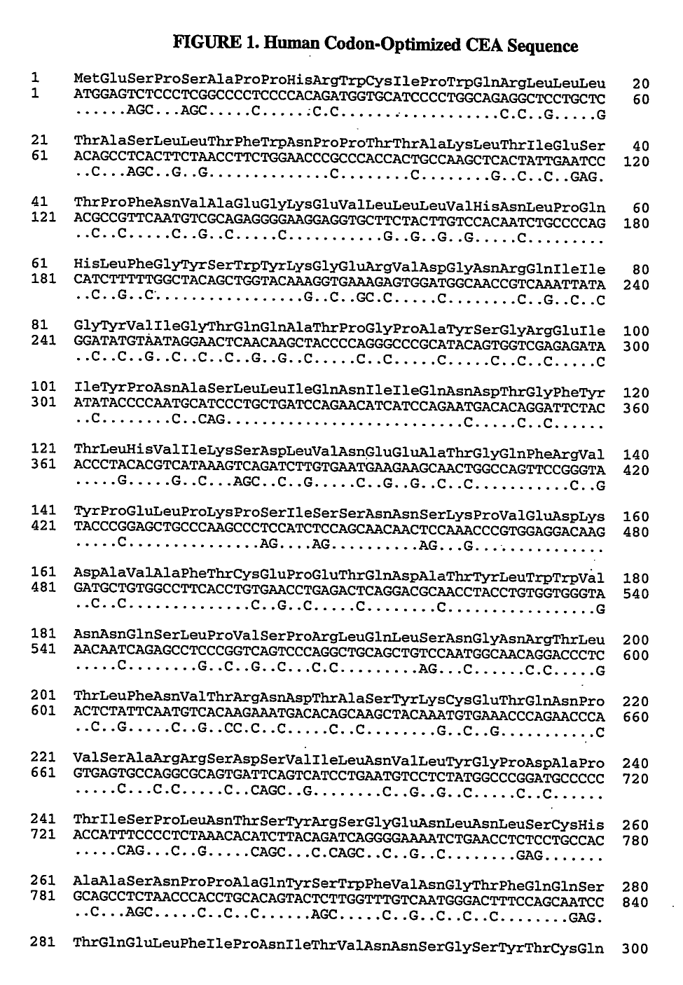

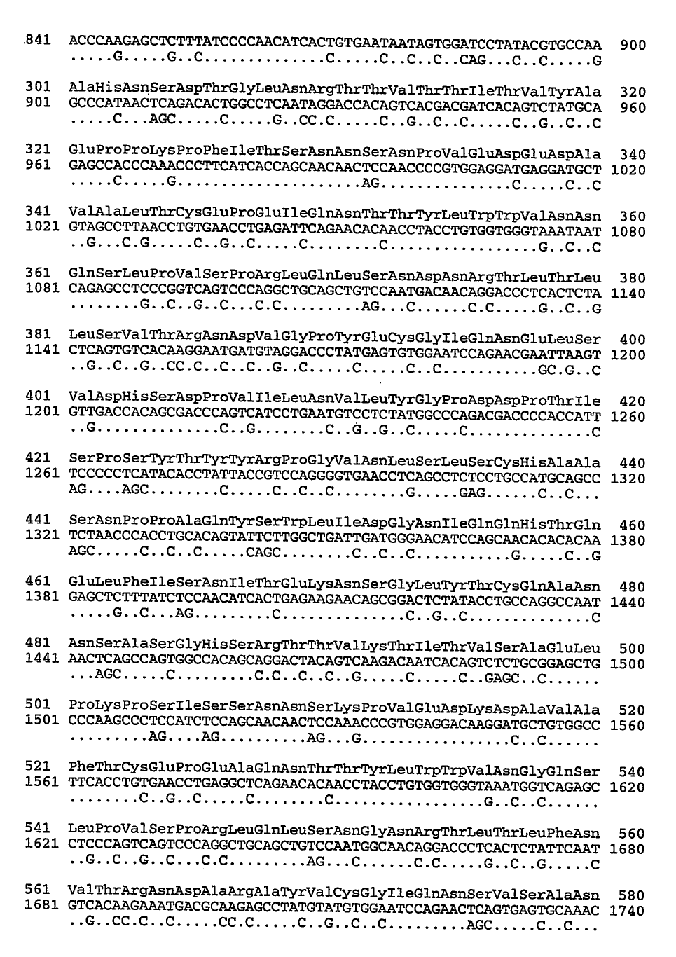

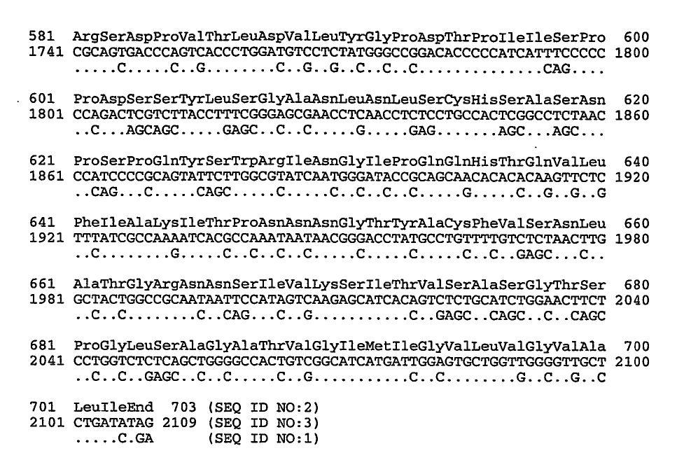

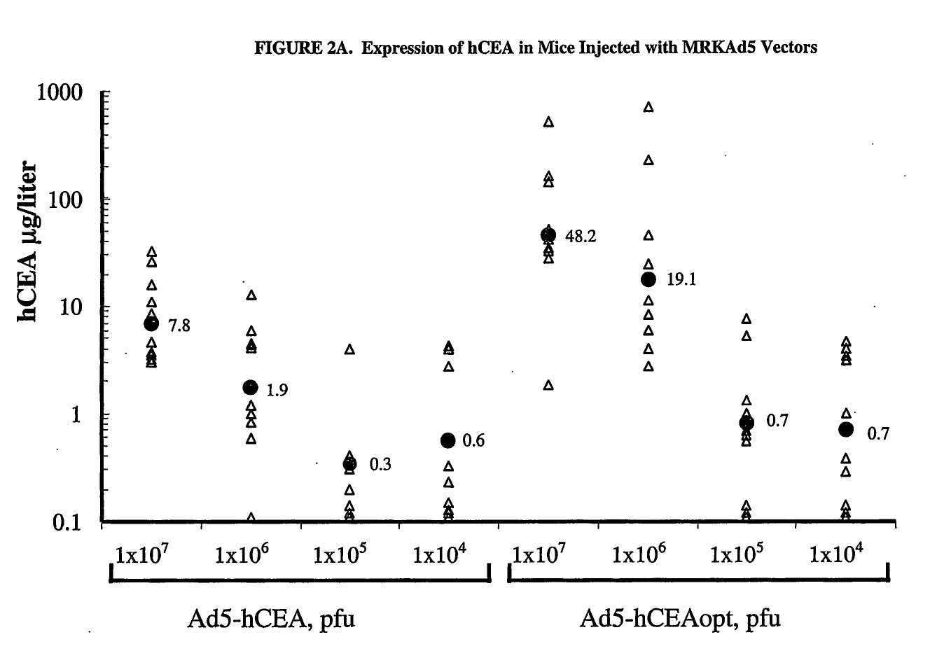

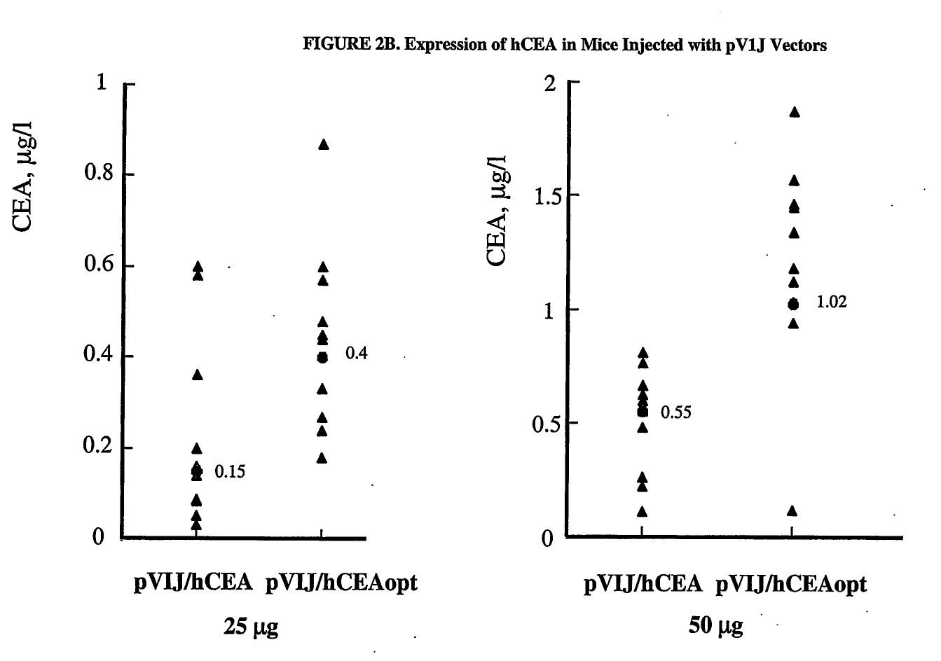

A preferred DNA molecule of the present invention comprises the nucleotide sequence disclosed herein as SEQ ID NO:1, shown in FIG. 1, which encodes the human CEA protein shown in FIG. 2 and set forth as SEQ ID NO:2.

A further preferred DNA molecule of the present invention comprises the nucleotide sequence disclosed herein as SEQ ID NO:15, shown in FIG. 10A, which encodes a human CEA variant that is deleted of its C-terminal anchoring sequence, as set forth in SEQ ID NO:16, and shown in FIG. 10B.

The present invention also includes biologically active fragments or mutants of SEQ ID NOs:1, which encode mRNA expressing human CEA proteins. Any such biologically active fragment and/or mutant will encode either a protein or protein fragment which at least substantially mimics the pharmacological properties of the hCEA protein, including but not limited to the hCEA protein as set forth in SEQ ID NO:2. Any such polynucleotide includes but is not necessarily limited to: nucleotide substitutions, deletions, additions, amino-terminal truncations and carboxy-terminal truncations. The mutations of the present invention encode mRNA molecules that express a functional hCEA protein in a eukaryotic cell so as to be useful in cancer vaccine development.

This invention also relates to synthetic codon-optimized DNA molecules that encode the hCEA protein wherein the nucleotide sequence of the synthetic DNA differs significantly from the nucleotide sequence of SEQ ID NO:1, but still encodes the hCEA protein as set forth in SEQ ID NO:2. Such synthetic DNAs are intended to be within the scope of the present invention. Therefore, the present invention discloses codon redundancy that may result in numerous DNA molecules expressing an identical protein. Also included within the scope of this invention are mutations in the DNA sequence that do not substantially alter the ultimate physical properties of the expressed protein. For example, substitution of valine for leucine, arginine for lysine, or asparagine for glutamine may not cause a change in the functionality of the polypeptide.

It is known that DNA sequences coding for a peptide may be altered so as to code for a peptide that has properties that are different than those of the naturally occurring peptide. Methods of altering the DNA sequences include but are not limited to site directed mutagenesis. Examples of altered properties include but are not limited to changes in the affinity of an enzyme for a substrate or receptor for a ligand.

The present invention also relates to hCEAopt fusion constructs, including but not limited to fusion constructs which express a portion of the human CEA protein linked to various markers, including but in no way limited to GFP (Green fluorescent protein), the MYC epitope, GST, and Fc. Any such fusion construct may be expressed in the cell line of interest and used to screen for modulators of the human CEA protein disclosed herein. Also contemplated are fusion constructs that are constructed to enhance the immune response to human CEA including, but not limited to: DOM and hsp70, and LTB.

The present invention further relates to recombinant vectors that comprise the synthetic nucleic acid molecules disclosed throughout this specification. These vectors may be comprised of DNA or RNA. For most cloning purposes, DNA vectors are preferred. Typical vectors include plasmids, modified viruses, baculovirus, bacteriophage, cosmids, yeast artificial chromosomes, and other forms of episomal or integrated DNA that can encode a hCEA protein. It is well within the purview of the skilled artisan to determine an appropriate vector for a particular gene transfer or other use.

An expression vector containing codon-optimized DNA encoding a hCEA protein may be used for high-level expression of hCEA in a recombinant host cell. Expression vectors may include, but are not limited to, cloning vectors, modified cloning vectors, specifically designed plasmids or viruses. Also, a variety of bacterial expression vectors may be used to express recombinant hCEA in bacterial cells if desired. In addition, a variety of fungal cell expression vectors may be used to express recombinant hCEA in fungal cells. Further, a variety of insect cell expression vectors may be used to express recombinant protein in insect cells.

The present invention also relates to host cells transformed or transfected with vectors comprising the nucleic acid molecules of the present invention. Recombinant host cells may be prokaryotic or eukaryotic, including but not limited to, bacteria such as E. coli, fingal cells such as yeast, mammalian cells including, but not limited to, cell lines of bovine, porcine, monkey and rodent origin; and insect cells including but not limited to Drosophila and silkworm derived cell lines. Such recombinant host cells can be cultured under suitable conditions to produce hCEA or a biologically equivalent fornl In a preferred embodiment of the present invention, the host cell is human. As defined herein, the term “host cell” is not intended to include a host cell in the body of a transgenic human being, human fetus, or human embryos.

As noted above, an expression vector containing DNA encoding a hCEA protein may be used for expression of hCEA in a recombinant host cell. Therefore, another aspect of this invention is a process for expressing a human CEA protein or protein variant in a recombinant host cell, comprising: (a) introducing a vector comprising a nucleic acid as set forth in SEQ ID NO:1 or SEQ ID NO:15 into a suitable human host cell; and, (b) culturing the host cell under conditions which allow expression of said human CEA protein or CEA protein variant.

Following expression of hCEA in a host cell, hCEA protein may be recovered to provide hCEA protein in active form Several hCEA protein purification procedures are available and suitable for use. Recombinant hCEA protein may be purified from cell lysates and extracts by various combinations of, or individual application of salt fractionation, ion exchange chromatography, size exclusion chromatography, hydroxylapatite adsorption chromatography and hydrophobic interaction chromatography. In addition, recombinant hCEA protein can be separated from other cellular proteins by use of an immunoaffinity column made with monoclonal or polyclonal antibodies specific for full-length hCEA protein, or polypeptide fragments of hCEA protein.

The nucleic acids of the present invention may be assembled into an expression cassette which comprises sequences designed to provide for efficient expression of the protein in a human cell. The cassette preferably contains a full-length codon-optimized hCEA gene, with related transcriptional and translations control sequences operatively linked to it, such as a promoter, and termination sequences. In a preferred embodiment, the promoter is the cytomegalovirus promoter without the intron A sequence (CMV), although those skilled in the art will recognize that any of a number of other known promoters such as the strong immunoglobulin, or other eukaryotic gene promoters may be used. A preferred transcriptional terminator is the bovine growth hormone terminator, although other known transcriptional terminators may also be used. The combination of CMV-BGH terminator is particularly preferred.

In accordance with this invention, the hCEAopt expression cassette is inserted into a vector. The vector is preferably an adenoviral vector, although linear DNA linked to a promoter, or other vectors, such as adeno-associated virus or a modified vaccinia virus, retroviral or lentiviral vector may also be used.

If the vector chosen is an adenovirus, it is preferred that the vector be a so-called first-generation adenoviral vector. These adenoviral vectors are characterized by having a non-functional E1 gene region, and preferably a deleted adenoviral E1 gene region. In some embodiments, the expression cassette is inserted in the position where the adenoviral E1 gene is normally located. In addition, these vectors optionally have a non-functional or deleted E3 region. It is preferred that the adenovirus genome used be deleted of both the E1 and E3 regions (ΔE1ΔE3). The adenoviruses can be multiplied in known cell lines which express the viral E1 gene, such as 293 cells, or PERC.6 cells, or in cell lines derived from 293 or PERC.6 cell which are transiently or stablily transformed to express an extra protein. For examples, when using constructs that have a controlled gene expression, such as a tetracycline regulatable promoter system, the cell line may express components involved in the regulatory system. One example of such a cell line is T-Rex-293; others are known in the art.

For convenience in manipulating the adenoviral vector, the adenovirus may be in a shuttle plasmid form. This invention is also directed to a shuttle plasmid vector which comprises a plasmid portion and an adenovirus portion, the adenovirus portion comprising an adenoviral genome which has a deleted E1 and optional E3 deletion, and has an inserted expression cassette comprising codon-optimized human CEA. In preferred embodiments, there is a restriction site flanking the adenoviral portion of the plasmid so that the adenoviral vector can easily be removed. The shuttle plasmid may be replicated in prokaryotic cells or eukaryotic cells.

In a preferred embodiment of the invention, the expression cassette is inserted into the pMRKAd5-HV0 adenovirus plasmid (See Emini et al., WO 02/22080, which is hereby incorporated by reference). This plasmid comprises an Ad5 adenoviral genome deleted of the E1 and E3 regions. The design of the pMRKAd5-HV0 plasmid was improved over prior adenovectors by extending the 5′ cis-acting packaging region further into the E1 gene to incorporate elements found to be important in optimizing viral packaging, resulting in enhanced virus amplification. Advantageously, this enhanced adenoviral vector is capable of maintaining genetic stability following high passage propagation.

Standard techniques of molecular biology for preparing and purifying DNA constructs enable the preparation of the adenoviruses, shuttle plasmids, and DNA immunogens of this invention.

It has been determined in accordance with the present invention that the synthetic cDNA molecule described herein (SEQ ID NO:1), which is codon-optimized for high-level expression in a human cell, is expressed with greater efficiency than the corresponding wild type sequence. Surprisingly, the codon optimized cDNA of hCEA breaks tolerance to hCEA more efficiently than the wild type sequence. Additionally, it was shown herein that hCEAopt is more immunogenic that hCEA and is more efficient in eliciing both cellular and humoral immune responses.

Therefore, the vectors described above may be used in immunogenic compositions and vaccines for preventing the development of adenocarcinomas associated with aberrant CEA expression and/or for treating existing cancers. The vectors of the present invention allow for vaccine development and commercialization by eliminating difficulties with obtaining high expression levels of exogenous CEA in successfully transformed host organisms. To this end, one aspect of the instant invention is a method of preventing or treating cancer comprising administering to a mammal a vaccine vector comprising a synthetic codon-optimized nucleic acid molecule, the synthetic codon-opized nucleic acid molecule comprising a sequence of nucleotides that encodes a human CEA protein as set forth in SEQ ID NO:2.

In accordance with the method described above, the vaccine vector may be administered for the treatment or prevention of cancer in any mammal. In a preferred embodiment of the invention, the mammal is a human.

Further, one of skill in the art may choose any type of vector for use in the treatment and prevention method described. Preferably, the vector is an adenovirus vector or a plasmid vector. In a preferred embodiment of the invention, the vector is an adenoviral vector comprising an adenoviral genome with a deletion in the adenovirus E1 region, and an insert in the adenovirus E1 region, wherein the insert comprises an expression cassette comprising: (a) a synthetic codon-optimized polynucleotide encoding a human CEA protein; and (b) a promoter operably linked to the polynucleotide.

The instant invention further relates to an adenovirus vaccine vector comprising an adenoviral genome with a deletion in the E1 region, and an insert in the E1 region, wherein the insert comprises an expression cassette comprising: (a) a synthetic codon-optimized polynucleotide encoding a human CEA protein; and (b) a promoter operably linked to the polynucleotide.

In a preferred embodiment of this aspect of the invention, the adenovirus vector is an Ad 5 vector.

In another preferred embodiment of the invention, the adenovirus vector is an Ad 6 vector.

In yet another preferred embodiment, the adenovirus vector is an Ad 24 vector.

In another aspect, the invention relates to a vaccine plasmid comprising a plasmid portion and an expression cassette portion, the expression cassette portion comprising: (a) a synthetic codon-optimized polynucleotide encoding a human CEA protein or variant thereof; and (b) a promoter operably linked to the polynucleotide.

In some embodiments of this invention, the recombinant adenovirus vaccines disclosed herein are used in various prime/boost combinations with a plasmid-based polynucleotide vaccine in order to induce an enhanced immune response. In this case, the two vectors are administered in a “prime and boost” regimen. For example the first type of vector is administered, then after a predetermined amount of time, for example, 2 weeks, 1 month, 2 months, six months, or other appropriate interval, a second type of vector is administered. Preferably the vectors carry expression cassettes encoding the same polynucleotide or combination of polynucleotides. In the embodiment where a plasmid DNA is also used, it is preferred that the vector contain one or more promoters recognized by mammalian or insect cells. In a preferred embodiment, the plasmid would contain a strong promoter such as, but not limited to, the CMV promoter. The synthetic human CEA gene or other gene to be expressed would be linked to such a promoter. An example of such a plasmid would be the mammalian expression plasmid V1Jns as described (J. Shiver et. al. in DNA Vaccines, M. Liu et al. eds., N.Y. Acad. Sci., N.Y., 772:198-208 (1996), which is herein incorporated by reference).

As stated above, an adenoviral vector vaccine and a plasmid vaccine may be administered to a vertebrate as part of a single therapeutic regime to induce an immune response. To this end, the present invention relates to a method of protecting a mammal from cancer comprising: (a) introducing into the mammal a first vector comprising: i) a synthetic codon-optimized polynucleotide encoding a human CEA protein or human CEA protein variant; and ii) a promoter operably linked to the polynucleotide; (b) allowing a predetermined amount of time to pass; and (c) introducing into the mammal a second vector comprising: i) a synthetic codon-optimized polynucleotide encoding a human CEA protein or human CEA protein variant; and ii) a promoter operably linked to the polynucleotide.

In one embodiment of the method of protection described above, the first vector is a plasmid and the second vector is an adenovirus vector. In an alternative embodiment, the first vector is an adenovirus vector and the second vector is a plasmid.

The instant invention further relates to a method of treating a mammal suffering from an adenocarcinoma comprising: (a) introducing into the mammal a first vector comprising: i) a synthetic codon-optimized polynucleotide encoding a human CEA protein or human CEA protein variant; and ii) a promoter operably linked to the polynucleotide; (b) allowing a predetermined amount of time to pass; and (c) introducing into the mammal a second vector comprising: i) a synthetic codon-optimized polynucleotide encoding a human CEA protein or human CEA protein variant; and ii) a promoter operably linked to the polynucleotide.

In one embodiment of the method of treatment described above, the first vector is a plasmid and the second vector is an adenovirus vector. In an alternative embodiment, the first vector is an adenovirus vector and the second vector is a plasmid.

The amount of expressible DNA or transcribed RNA to be introduced into a vaccine recipient will depend partially on the strength of the promoters used and on the immunogenicity of the expressed gene product. In general, an immunologically or prophylactically effective dose of about 1 ng to 100 mg, and preferably about 10 μg to 300 μg of a plasmid vaccine vector is administered directly into muscle tissue. An effective dose for recombinant adenovirus is approximately 10⁶-10¹² particles and preferably about 10⁷-10¹¹ particles. Subcutaneous injection, intradermal introduction, impression though the skin, and other modes of administration such as intraperitoneal, intravenous, or inhalation delivery are also contemplated. It is also contemplated that booster vaccinations may be provided. Parenteral administration, such as intravenous, intramuscular, subcutaneous or other means of administration with adjuvants such as interleukin 12 protein, concurrently with or subsequent to parenteral introduction of the vaccine of this invention is also advantageous.

The vaccine vectors of this invention may be naked, i.e., unassociated with any proteins, adjuvants or other agents which impact on the recipient's immune system. In this case, it is desirable for the vaccine vectors to be in a physiologically acceptable solution, such as, but not limited to, sterile saline or sterile buffered saline. Alternatively, it may be advantageous to administer an immunostimulant, such as an adjuvant, cytokine, protein, or other carrier with the vaccines or immunogenic compositions of the present invention. Therefore, this invention includes the use of such immunostimulants in conjunction with the compositions and methods of the present invention. An immunostimulant, as used herein, refers to essentially any substance that enhances or potentiates an immune response (antibody and/or cell-mediated) to an exogenous antigen. Said immunostimulants can be administered in the form of DNA or protein. Any of a variety of immunostimulants may be employed in conjunction with the vaccines and immunogenic compositions of the present inventions, including, but not limited to: GM-CSF, IFNα, tetanus toxoid, IL12, B7.1, LFA-3 and ICAM-1. Said inmmunostimulants are well-known in the art. Agents which assist in the cellular uptake of DNA, such as, but not limited to calcium ion, may also be used. These agents are generally referred to as transfection facilitating reagents and pharmaceutically acceptable carriers. Those of skill in the art will be able to determine the particular immunostimulant or pharmaceutically acceptable carrier as well as the appropriate time and mode of administration.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLE 1

### Human CEA Optimized Codon Sequence

The entire hCEAopt coding sequence was synthesized and assembled by BIONEXUS (Oakland, Calif.). The hCEAopt cDNA, which carries an optimized Kozak sequence at its 5′-end, was constructed using oligonucleotides assembled by PCR. The assembled cDNA was inserted into the pCR-Blunt vector (Invitrogen, Carlsbad, Calif.), yielding pCR-hCEAopt. The integrity of the hCEAopt cDNA was determined by sequencing of both strands.

### EXAMPLE 2

### Plasmid Constructs and Adenovirus Vectors

pV1J/hCEAopt: Plasmid pCR-hCEAopt was digested with EcoRI for 1 hr at 37° C. The resulting 2156 bp insert was purified and cloned into the EcoRI site of plasmid pV1JnsB (Montgomery, et al., DNA Cell Biol., 12(9):777-83(1993)).

pV1J/hCEA: Plasmid pCI/hCEA (Song et al. Regulation of T-helper-1 versus T-helper-2 activity and enhancement of tumour immunity by combined DNA-based vaccination and nonviral cytokine gene transfer. Gene Therapy 7: 481-492 (2000)) was digested with EcoRI. The resulting 2109 bp insert was cloned into the EcoRI site of plasmid pV1JnsA (Montgomery et al., supra).

Ad5/hCEAopt: Plasmid pCR-hCEAopt was digested with EcoRI. The resulting 2156 bp insert was purified and cloned into the EcoRI of the polyMRK-Ad5 shuttle plasmid (See Emini et al., WO 02/22080, which is hereby incorporated by reference).

Ad5/CEA: The shuttle plasmid pMRK-hCEA for generation of Ad5 vector was obtained by digesting plasmid pDelta1sp1B/hCEA with SspI and EcoRV. The 9.52 kb fragment was then ligated with a 1272 bp BglII-BamHI restricted, Klenow treated product from plasmid polyMRK. A PacI/StuI fragment from pMRK-hCEA and pMRK-hCEAopt containing the expression cassette for hCEA and E1 flanking Ad5 regions was recombined to ClaI linearized plasmid pAd5 in BJ5183 E. coli cells. The resulting plasmids were pAd5-hCEA and pAd5-hCEAopt, respectively. Both plasmids were cut with PacI to release the Ad inverted terminal repeats (ITR) and transfected in PerC-6 cells. Ad5 vectors amplification was carried out by serial passage. MRKAd5/hCEA and MRKAd5/hCEAopt were purified through standard CsCl gradient purification and extensively dialyzed against A105 buffer (5 mM Tris-Cl pH 8.0, 1 mM MgCl₂, 75 mM NaCal, 5% Sucrose, 0.005 Tween 20).

### EXAMPLE 3

### CEA Expression and Detection

Expression of hCEA by the plasmid and Ad vectors was monitored by Western blot analysis. Plasmids were transfected in HeLa cells or PerC.6 cells with Lipofectamine 2000 (Life Technologies, Carlsbad, Calif.). Adenovirus infections of PerC.6 cells were performed in serum free medium for 30 min at 37° C., then fresh medium was added. After 48 hr incubation, whole cell lysates and culture supernatant were harvested The CEA protein present in the cell lysates was detected by Western blot analysis using a rabbit polyclonal antiserum. The protein was detected as a 180-200 kDa band. The secreted CEA was detected in the cell supernatants and in peripheral blood of injected mice (3 days post injection) using the Direct Elisa CEA Kit (DBC-Diagnostics Biochem Canada Inc., Ontario, Canada).

### EXAMPLE 4

### Mice Immunization

Female C57BL/6 mice (H-2^{b}) were purchased from Charles River (Lecco, Italy). CEA.tg mice (H-2^{b}) were provided by J. Primus (Vanderbilt University) and kept in standard conditions. Fifty micrograms of plasmid DNA were electroinjected in a 50 μl volume in mice quadriceps as previously described (Rizzuto et al. Proc. Natl. Acad. Sci. U.S.A. 96(11): 6417-22 (1999)). Ad injections were carried out in mice quadriceps in 50 μl volume. Humoral and cell mediated immune response were analyzed at the indicated time.

### EXAMPLE 5

### Codon Optimized cDNA of hCEA Significantly Increased hCEA Expression

A synthetic gene of human CEA (hCEAopt) was designed to incorporate human-preferred (humanized) codons for each amino acid (hereinafter aa) residue. The codon optimized cDNA was modified to maintain 76.8% nucleotide identity to the original clone (see FIG. 1). The codon optimized cDNAs were cloned into the pV1J vectors (Montgomery et al., supra), placing in front a Kozak optimized sequence (5′-GCCGCCACC-3′, SEQ ID NO:13) and under the control of the human cytomegalovirus (CMV)/intron A promoter plus the bovine growth hormone (BGH) termination signal. The construct was named pV1J/hCEAopt (see EXAMPLE 2). Additionally, an Adenovirus type 5 vector was constructed carrying the hCEAopt sequence flanked by the CMV/intron A promoter and the BGH termination signal (Ad5/hCEAopt). For comparison, the equivalent plasmid and Ad5 vectors were constructed carrying the wild type hCEA sequence yielding pV1J/hCEA and Ad5/hCEA. Similar to those containing the codon optimized cDNA, these vectors carry the wild type gene under the control of the CMV/int A promoter with BGH termination signal.

Western blot analysis of HeLa cells transfected with plasmid pV1J/hCEAopt yielded a protein with large molecular mass (180-200 kDa) that was indistinguishable in size from that detected in cells transfected with construct pV1J/hCEA. Similarly, no apparent differences could be detected in the size of the protein detected in PerC-6 cell lysates that had been infected with Ad5/hCEA or Ad5H7hCEAopt (data not shown).

To compare the efficiency of expression of the hCEAopt to that of hCEA, groups of 10 C57BLU6 mice were injected into the quadriceps with different doses of the Ad5/hCEAopt vector ranging from 1×10⁷ to 1×10⁴ pfu. Three days post injection, CEA protein levels were determined and compared to those of control groups that had been injected with the same doses of Ad5/hCEA. A sixfold increase in the geometric mean values of hCEA levels was observed upon injection of 1×10⁷ pfu of Ad/hCEAopt (48.2 μg/1) relative to the Ad5/hCEA injected mice, whereas a tenfold increase in protein level was observed upon injection of 1×10⁶ pfu of the same virus (19.1 μg/l) (FIG. 2A). In contrast, injection of lower doses of Ad5/hCEAopt did not result in a substantial increase in circulating CEA levels as compared to Ad5/hCEA. The enhancement of CEA protein levels was also noted, albeit to a lower extent, upon electroinjection of 25 or 50 μg of plasmid pV1J/hCEAopt relative to pV1J/hCEA (FIG. 2B). Thus, these results indicate that, independently of the gene transfer vehicle utilized, the codon optimized cDNA is expressed with greater efficiency than the corresponding wild type sequence.

### EXAMPLE 6

### IFN-γ ELISPOT Assay

Ninety-six wells MAIP plates (Millipore, Bedford, Mass.) were coated with 100 μl/ well of purified rat anti-mouse IFN-γ (IgG1, clone R4-6A2, Pharmingen, San Diego, Calif.) diluted to 2.5 μg/ml in sterile PBS. After washing with PBS, blocldng of plates was carried out with 200 μl/well of R10 medium for 2 hrs at 37° C.

Splenocytes were obtained by removing the spleen from the euthanized mice in a sterile manner. Spleen disruptionwas carried out by grating the dissected spleen on a metal grid. Red blood cells were removed by osmotic lysis by adding 1 ml of 0.1×PBS to the cell pellet and vortexing no more than 15 seconds. One ml of 2×PBS was then added and the volume was brought to 4 ml with 1×PBS. Cells were pelleted by centrifugation at 1200 rpm for 10 min at room temp., and the pellet was resuspended in 1 ml R10 medium. Viable cells were counted using Turks staining.

Splenocytes were plated at 5×10⁵ and 2×10⁵ cells/well in duplicate and incubated for 20 h at 37° C. with 1 μg/ml suspension of each peptide. Concanavalin A (ConA) was used as positive internal control for each mouse at 5 μg/ml. After washing with PBS, 0.05% Tween 20, plates were incubated O/N at 4° C. with 50 μl/well of biotin-conjugated rat anti-mouse IFNγ (RatIgG1, clone XMG 1.2, PharMingen) diluted to 1:2500 in Assay buffer. After extensive washing, plates were developed by adding 50 μl/well NBT/B-CIP (Pierce Biotechnology Inc., Rockford, Ill.) until development of spots was clearly visible. The reaction was stopped by washing plates thoroughly with distilled water. Plates were air dried and spots were then counted using an automated ELISPOT reader.

### EXAMPLE 7

### Intracellular Cytokine Staining

One to two million mouse splenocytes or PBMC in 1 ml RPMI 10% FCS were incubated with a pool of peptides (5-6 μg/ml final concentration of each peptide) and brefeldin A (1 μg/ml; BD Pharmingen cat #555028/2300kk) and 5% CO₂ for 12-16 hours at 37° C. Cells were then washed with FACS buffer (PBS 1% FBS, 0.01% NaN₃) and incubated with purified anti-mouse CD16/CD32 Fc block (BD Pharmingen cat # 553142) for 15 min at 4° C. Cells were then washed and stained with surface antibodies: CD4-PE conjugated anti-mouse (BD Pharmingen, cat.# 553049), PercP CD8 conjugated anti mouse (BD Pharmingen cat# 553036) and APC-conjugated anti-mouse CD3e (BD Pharmingen cat#553066) for 30 minutes at room temperature in the dark. After the washing, cells were fixed and permeabilized with Cytofix-Cytoperm Solution (BD Pharmingen cat #555028/2300kk) for 20 min at 4° C. in the dark. After washing with PermWash Solution (BD Pharmingen cat #555028/2300kk) cells were incubated with the IFNγ-FTTC antibodies (BD Pharmingen). Cells were then washed, fixed with formaldehyde 1% in PBS and analyzed on a FACS-Calibur flow cytometer, using CellQuest software (Becton Dickinson, San Jose, Calif.).

### EXAMPLE 8

### Identification and Characterization of Epitope Containing Peptides for Direct Enumeration of CEA-specific T Cells

To better characterize the immune response elicited upon genetic vaccination against CEA in mice, ELISPOT analysis was carried out on C57BL/6 and BALB/c mice to identify CD4+ and CD8+ CEA specific epitopes. To this end, different immunization modalities were compared to generate highly immunized mice that could be utilized to identify responses to individual peptides that cover the entire protein. In view of recent reports that indicate that high levels of cellular immunity can be induced against viral and bacterial antigens by utilizing plasmid DNA prime-Ad boost modality, the same immunization protocol was employed in this study. Mice were immunized intramuscularly by different regimens: i) two doses of 1×10⁹ vp of Ad/hCEA (Ad/Ad), ii) two doses of plasmid pV1J/hCEA (DNA/DNA) and iii) a dose of plasmid DNA followed by Ad/hCEA (DNA/Ad). The immunizations were two weeks apart.

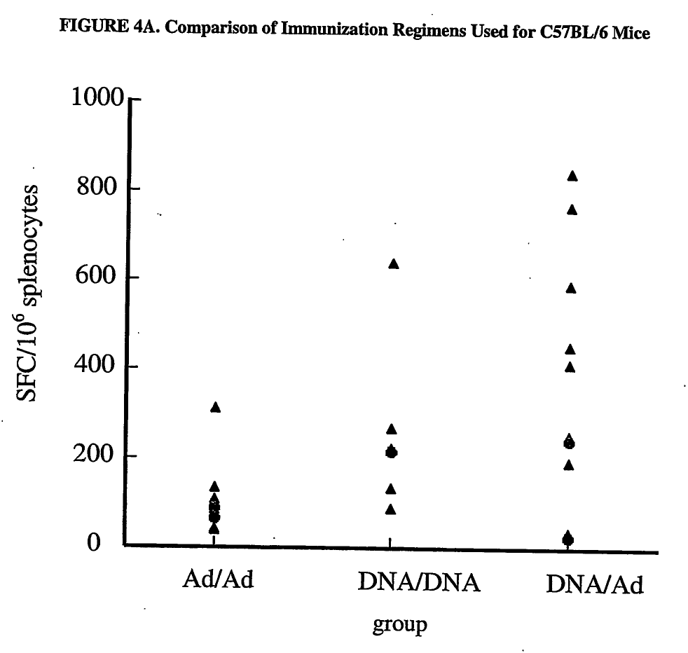

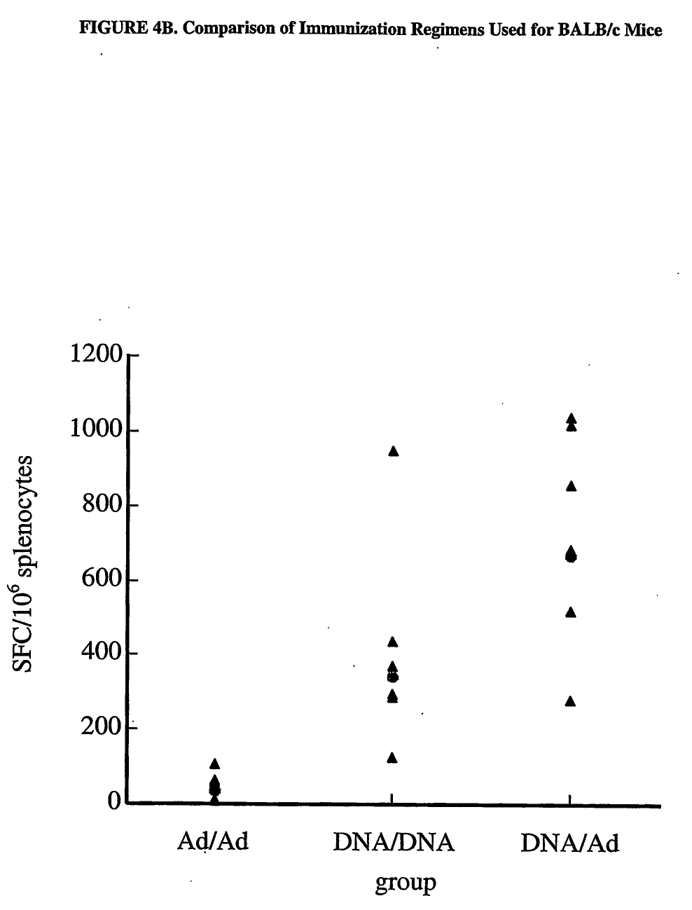

The cellular immunity elicited by the different immunization regimes was measured by ELISPOT assay 2 weeks after the boost. To compare the immunogenic efficiency of the different vaccination regimens, a pool of 15 mer peptides overlapping by 11 aa and covering aa 497-703 (pool D) were used to stimulate antigen specific cytokine secretion from splenocytes. The most vigorous responses, indicated by the higher geometric mean values of the SFC, were observed in C57BL/6 and BALB/c mice from the DNA/Ad injected group (FIG. 4). Thus, this regimen was utilized to further analyze the immune response.

To determine whether the immune response was equally distributed across the entire CEA protein, splenocytes from immunized C57BL/6 and BALB/c mice were stimulated in vitro with one of four pools of 15-mer peptides that collectively encompass the entire protein sequence. Each pool consisted of peptides 15 amino acids long that overlap by 11 residues. Lyophilized hCEA peptides were purchased from Bio-Synthesis (Lewisville, Tex.) and resuspended in DMSO at 40 mg/ml. In addition to pool D, pools A (aa 1 to 147), B (aa 137 to 237), and C (aa 317 to 507) were used in this study. Final concentrations were the following: pool A=1.2 mg/ml, pool B=0.89 mg/ml, pool C=0.89 mg/ml, pool D=0.8 mg/ml. Peptides were stored at −80° C.

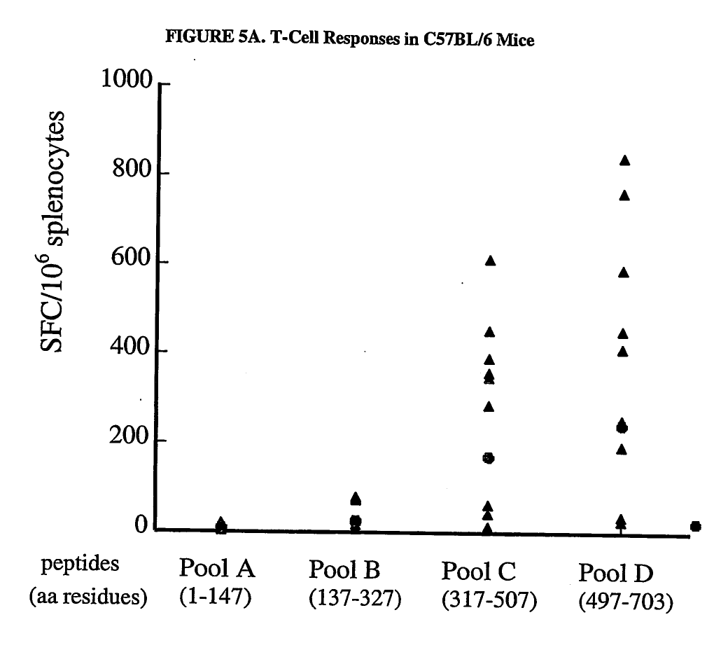

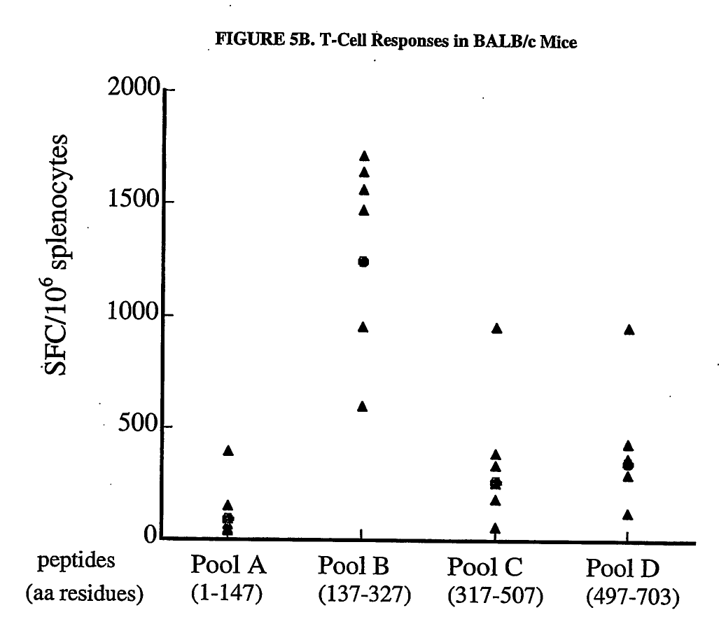

The immune response elicited by the DNA/Ad vaccination regimen in C57BL/6 mice was primarily biased towards the C-terminal region of the protein (see FIG. 5A). Significant SFC values were obtained with peptide pool C and D (geometric mean values: 170 and 244 SFC/10⁶ splenocytes, respectively), whereas pool A and B yielded much lower values (10 and 27 SFC/10⁶ splenocytes, respectively). In contrast, the immune response in BALB/c mice was highest with pool B (geometric mean value: 1236 SFC/10⁶ splenocytes), although pool A, C, and D showed significant SFC values (93, 263, and 344 respectively) (FIG. 5B). No responses against a pool of unrelated peptides were noted in both groups of mice (data not shown).

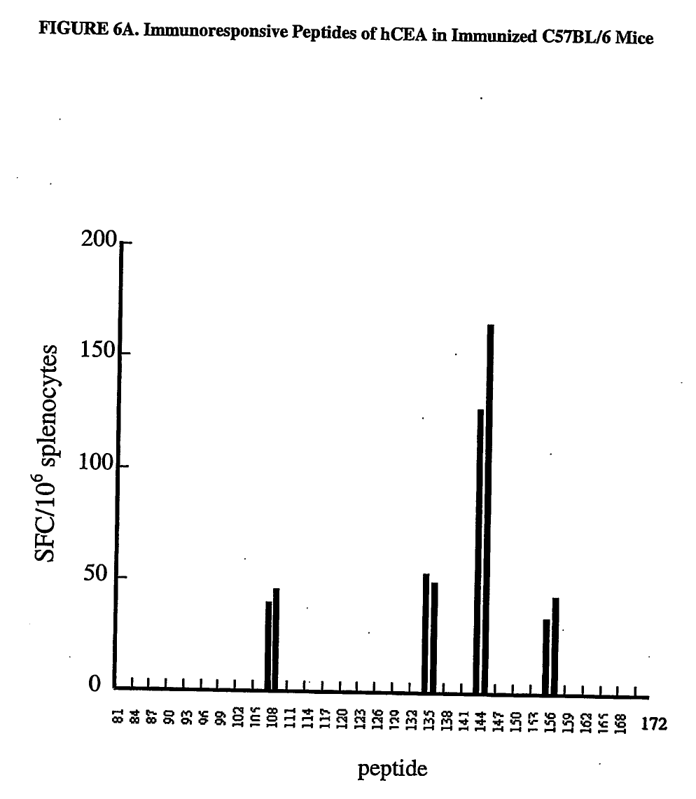

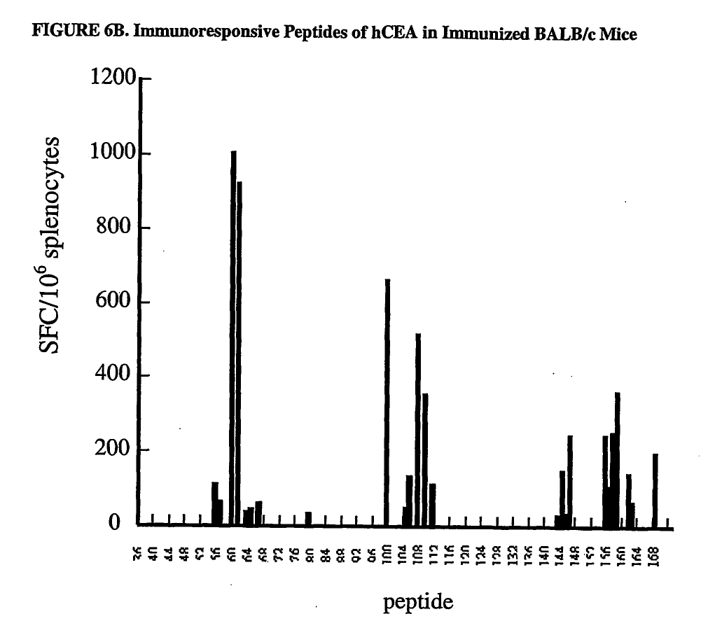

To identify the individual peptides present in the peptide pools that elicit the responses, spleens from 4 mice immunized with the DNA/Ad vaccination regimen were analyzed in a IFNγ-ELISPOT assay against each of the individual peptides comprising the pools against which a significant immune response had been observed. Splenocytes from C57BL/6 mice were tested against peptides 80 to 173 included in pool C and D. Splenocytes from BALB/c mice were tested against peptides 35 to 173 that comprise pools B, C, and D. CEA specific responses in C57BL/6 mice were mapped to four pairs of 15-mer peptides that had overlapping sequences (aa 431 to 435 and 425 to 439; 529 to 543, and 533 to 547; 565 to 579, and 569 to 593; 613 to 627 and 617 to 631) (FIG. 6A). The immune response to CEA in BALB/c mice was mapped to 22 different peptides, 17 of which have overlapping sequences (aa 213 to 227, and 213 and 227; 229 to 243, and 233 to 247; 409 to 423 and 413 to 427; 421 to 435 and 425 to 439; 565 to 579 and 569 to 583; 573 to 587; 613 to 627 and 617 to 631, and 621 to 635 and 625 to 639; 637 to 651 and 641 to 655) (FIG. 6B).

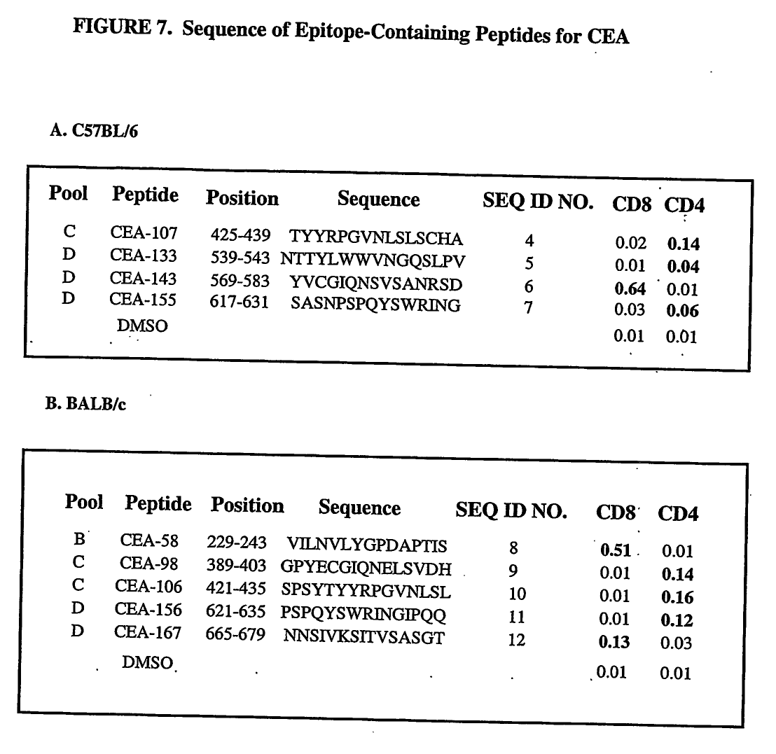

To define the T-cell specificity of the epitopes contained within the selected peptides, IFNγ intracellular staining assay was carried out on splenocytes from injected mice. The results obtained are shown in FIG. 7. The data indicate that CD8+ and CD4+ specific epitopes have been identified for both C57BL/6 and BALB/c which can be used to quantify circulating levels of T-lymphocytes.

### EXAMPLE 9

### Codon Optimized hCEA cDNA Breaks Tolerance in hCEA Transgenic Mice

To determine whether the enhanced immunogenic properties of the codon optimized cDNA of hCEA would break tolerance to human CEA more efficiently, hCEA transgenic mice were immunized with vectors carrying either the wild type or the codon optimized hCEA sequences. These transgenic mice carry the entire human CEA gene plus flanking sequences and express the hCEA protein in the cecum and colon. Thus, this mouse line is a useful model for studying the safety and efficacy of immunotherapy strategies directed against this tumor self antigen (Clarke et al. Mice transgenic for human CEA as a model for immunotherapy. Cancer Res. 58(7): 1469-77 (1998)).

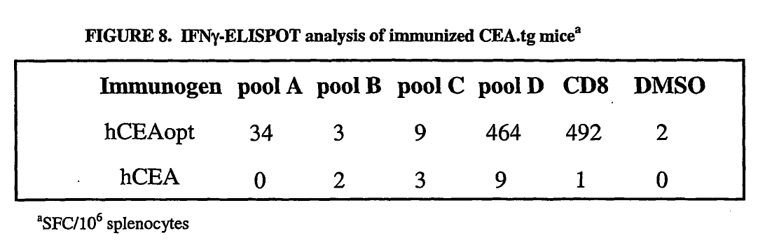

As a first test, groups of 5 to 10 transgenic mice were subjected to four electroinjections of 50 μg plasmid DNA followed by a final injection of 1×10¹⁰ pp of Adenovirus. The immune response to hCEA was analyzed by IFNγ-ELISPOT assay on pooled splenocytes from 4 injected mice. The immune response to hCEA was detected only with the splenocytes from the mice immunized with the hCEAopt cDNA (see FIG. 8). The immune response was detected with peptides 143 and pool D, suggesting that immunization had elicited a significant CD8+ response to the C-terminal epitopes.

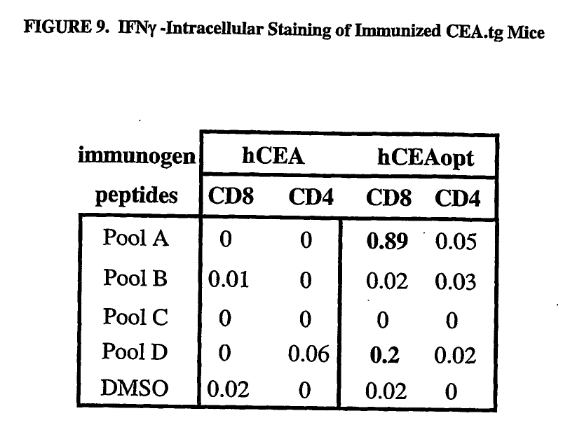

The enhanced immunogenicity of the codon optimized cDNA of hCEA was also tested in transgenic mice using two injections of 1×10¹⁰ pp of Adenovirus vectors two weeks apart. The CEA-specific immune response was measured by IFNγ intracellular staining on pooled PBMC from 4 immunized mice. The immune response to hCEA was detected only in mice immunized with Ad/CEAopt (FIG. 9). As observed with the DNA plus Ad cohort, induction of CD8+ T-cells was detected with peptide pool D; however, a significant CD8+ response was also noted with peptide pool A. Thus, these results indicate that the codon optimized cDNA of hCEA is more immunogenic and breaks tolerance to hCEA more efficiently than the wild type sequence.

### EXAMPLE 10

### Antibodies Detection and Titration

Sera for antibody titration were obtained by retro-orbital bleeding. ELISA plates (Nunc maxisorp™) were coated with 100 ng/well with CEA protein (highly pure CEA; Fitzgerald Industries International Inc., Concord Mass.), diluted in coating buffer (50 mM NaHCO₃, pH 9.4) and incubated O/N at 4° C. Plates were then blocked with PBS containing 5% BSA for 1 hr at 37° C. Mouse sera were diluted in PBS 5% BSA (dilution 1/50 to evaluate seroconversion rate; dilutions from 1:10 to 1:31, 2150 to evaluate titer). Pre-immune sera were used as background. Diluted sera were incubated O/N at 4° C. Washes were carried out with PBS 1% BSA, 0.05% Tween 20. Secondary antibody (goat anti-mouse, IgG Peroxidase, Sigma) was diluted 1/2000 in PBS, 5% BSA and incubated 2-3 hr at room temp. on a shaker. After washing, plates were developed with 100 μl/well of TMB substrate (Pierce Biotechnology, Inc., Rockford, Ill.). The reaction was stopped with 25 μl/well of 1M H₂SO₄ solution and plates were read at 450 nm/620 nm. Anti-CEA serum titers were calculated as the reciprocal limiting dilution of serum producing an absorbance at least 3-fold greater than the absorbance of autologous pre-immune serum at the same dilution.

### EXAMPLE 11

### Increased Immunogenicity of hCEAopt

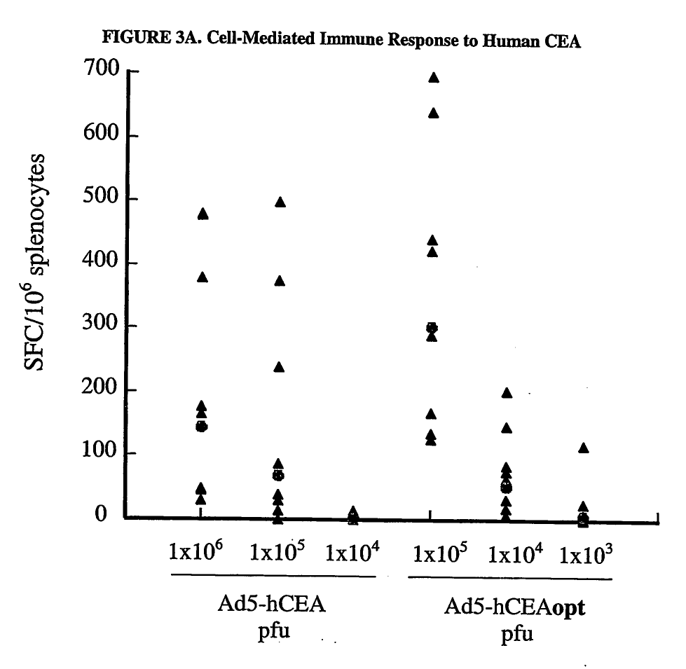

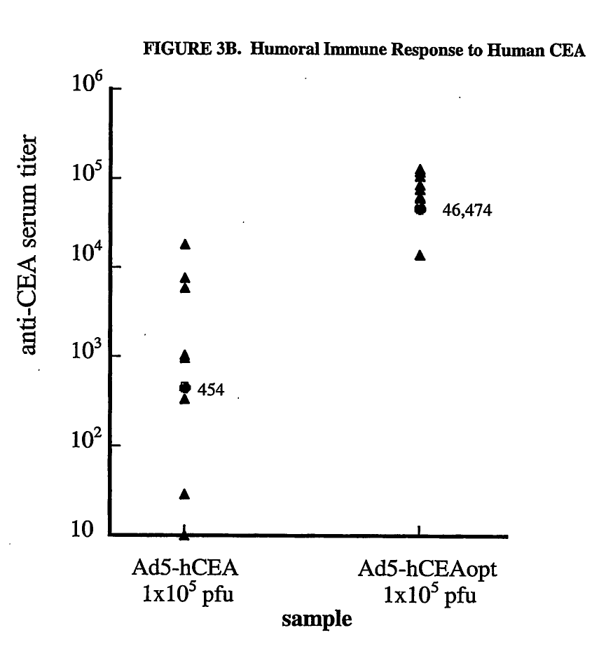

To examine in vivo immune responses induced by the wild type and codon optimized CEA expression vectors, C57BL/6 mice were immunized intramuscularly with different doses of Ad5/hCEAopt ranging from 1×10⁵ to 1×10³ pfu. As comparison, groups of 8 to 10 mice were immunized with Ad5/hCEA in doses ranging from 1×10⁶ to 1×10⁴ pfu. Mice were subjected to two injections three weeks apart. Two weeks after the second immunization, splenocytes were isolated from each mouse. To quantify the IFNγ secreting CEA-specific CD8 T-cell precursor frequencies generated by the Adenovirus mediated immunization, the ELISPOT assay for the H-2^{b} restricted T-cell epitope CGIQNSVSA (SEQ ID NO:14, see below) was used. Imunization with 1×10⁴ pfu elicited a measurable immune response yielding 53 IFNγ spot forming cells (SFC, geometric mean value) specific for the CGIQNSVSA epitope (SEQ ID NO:14), whereas injection of 1×10³ pfu elicited negligible SFC values (FIG. 3A). The SFC increased to 302 in the group immunized with 1×10⁵ pfu of Ad/hCEAopt. In contrast, at least 1×10⁵ pfu of Ad5/hCEA were necessary to elicit a significant CD8 T-cell precursor frequencies that increased to 168 SFC in the mouse group immunized with a dose of 1×10⁶ pfu. No peptide-specific IFNγ SFC were detected in the Ad5immunized mice (data not shown).

Sera from mice immunized with 1×10⁵ pfu of each hCEA Adenovirus vector were tested in ELISA using the purified hurnan CEA protein as substrate (FIG. 3B). CEA-specific antibody titer in Ad5/hCEAopt immunized mice was detected in all immunized mice and the geometric mean value of the Ab titer was 46,474. In contrast, the Ad5/hCEA immunized group showed an approximately 100 fold lower geometric mean titer of CAA-specific antibody (454). Thus, these results demonstrate that the codon optired cDNA of CEA is more efficient in eliciting an cellular and humoral immune response.

### EXAMPLE 12

### Statistical Analysis

Where indicated, results were analyzed by the Student t test. A p value <0.05 was considered significant.

1. A synthetic nucleic acid molecule comprising a sequence of nucleotides that encodes a human carcinoembryonic antigen (CEA) protein as set forth in SEQ ID NO:2, the synthetic nucleic acid molecule being codon-optimized for high level expression in a human cell.

2. The synthetic nucleic acid molecule of claim 1 wherein the nucleic acid is DNA.

3-4. (canceled)

5. The synthetic nucleic acid molecule of claim 1 wherein the sequence of nucleotides comprises the sequence of nucleotides set forth in SEQ ID NO:1.

6. A vector comprising the nucleic acid molecule of claim 1.

7. A host cell comprising the vector of claim 6.

8. A process for expressing a human carcinoembryonic antigen (CEA) protein in a recombinant host cell, comprising:
(a) introducing a vector comprising the nucleic acid of claim 1 into a suitable host cell; and,
(b) culturing the host cell under conditions which allow expression of said human CEA protein.

9. A method of preventing or treating cancer comprising administering to a human a vaccine vector comprising a synthetic codon-optimized nucleic acid molecule, the nucleic acid molecule comprising a sequence of nucleotides that encodes a human carcinoembryonic antigen (hCEA) protein as set forth in SEQ ID NO:2 or a CEA protein variant as set forth in SEQ ID NO:16.

10. (canceled)

11. A method according to claim 9 wherein the vector is an adenovirus vector or a plasmid vector.

12. A method according to claim 9 wherein the vector is an adenoviral vector comprising an adenoviral genome with a deletion in the adenovirus E1 region, and an insert in the adenovirus E1 region, wherein the insert comprises an expression cassette comprising:
(a) a codon-optimized polynucleotide encoding a human CEA protein or variant thereof; and
(b) a promoter operably linked to the polynucleotide.

13. A method according to claim 9 wherein the vector is a plasmid vaccine vector, which comprises a plasmid portion and an expressible cassette comprising
(a) a codon-optimized polynucleotide encoding a human CEA protein or variant thereof; and
(b) a promoter operably linked to the polynucleotide.

14. An adenovirus vaccine vector comprising an adenoviral genome with a deletion in the E1 region, and an insert in the E1 region, wherein the insert comprises an expression cassette comprising:
(a) a codon-optimized polynucleotide encoding a human CEA protein or variant thereof; and
(b) a promoter operably linked to the polynucleotide.

15. An adenovirus vector according to claim 14 which is an Ad 5 vector or an Ad 24 vector.

16. An adenovirus vector according to claim 14 which is an Ad 6 vector.

17. (canceled)

18. A vaccine plasmid comprising a plasmid portion and an expression cassette portion, the expression cassette portion comprising:
(a) a codon-optimized polynucleotide encoding a human CEA protein or variant thereof; and
(b) a promoter operably linked to the polynucleotide.

19. A method of protecting a mammal from cancer comprising:
(a) introducing into the mammal a first vector comprising:
(i) a codon-optimized polynucleotide encoding a human carcinoembryonic antigen (CEA) protein or variant thereof; and
(ii) a promoter operably linked to the polynucleotide;
(b) allowing a predetermined amount of time to pass; and
(c) introducing into the mammal a second vector comprising:
(i) a codon-optimized polynucleotide encoding a human CEA protein or variant thereof; and
(ii) a promoter operably linked to the polynucleotide.

20. A method according to claim 19 wherein the first vector is a plasmid and the second vector is an adenovirus vector.

21. A method according to claim 19 wherein the first vector is an adenovirus vector and the second vector is a plasmid.

22. A method of treating a mammal suffering from a colorectal carcinoma comprising:
(a) introducing into the mammal a first vector comprising:
(i) a codon-optimized polynucleotide encoding a human CEA protein or variant thereof; and
(ii) a promoter operably linked to the polynucleotide;
(b) allowing a predetermined amount of time to pass; and
(c) introducing into the mammal a second vector comprising:
(i) a codon-optimized polynucleotide encoding a human CEA protein or variant thereof; and
(ii) a promoter operably linked to the polynucleotide.

23. A method according to claim 22 wherein the first vector is a plasmid and the second vector is an adenovirus vector.

24. A method according to claim 22 wherein the first vector is an adenovirus vector and the second vector is a plasmid.

25. A synthetic nucleic acid molecule comprising a sequence of nucleotides that encodes a human carcinoembryonic antigen (CEA) protein variant as set forth in SEQ ID NO:16, the synthetic nucleic acid molecule being codon-optimized for high level expression in a human cell.

26. The synthetic nucleic acid molecule of claim 25 wherein the sequence of nucleotides comprises the sequence of nucleotides set forth in SEQ ID NO:15.

27. A vector comprising the nucleic acid molecule of claim 25.

28. A host cell comprising the vector of claim 27.


SEQUENCE LISTING (16 sequences)

SEQ ID NO: 1 - DNA, 2109 bp, Artificial Sequence
  hCEAopt
atggagagccccagcgcccccccccaccgctggtgcatcccctggcagcgcctgctgctgaccgccagcctgctgaccttctggaacccccccaccaccgccaagctgaccatcgagagcacccccttcaacgtggccgagggcaaggaggtgctgctgctggtgcacaacctgccccagcacctgttcggctacagctggtacaagggcgagcgcgtggacggcaaccgccagatcatcggctacgtgatcggcacccagcaggccacccccggccccgcctacagcggccgcgagatcatctaccccaacgccagcctgctgatccagaacatcatccagaacgacaccggcttctacaccctgcacgtgatcaagagcgacctggtgaacgaggaggccaccggccagttccgcgtgtaccccgagctgcccaagcccagcatcagcagcaacaacagcaagcccgtggaggacaaggacgccgtggccttcacctgcgagcccgagacccaggacgccacctacctgtggtgggtgaacaaccagagcctgcccgtgagcccccgcctgcagctgagcaacggcaaccgcaccctgaccctgttcaacgtgacccgcaacgacaccgccagctacaagtgcgagacccagaaccccgtgagcgcccgccgcagcgacagcgtgatcctgaacgtgctgtacggccccgacgcccccaccatcagccccctgaacaccagctaccgcagcggcgagaacctgaacctgagctgccacgccgccagcaacccccccgcccagtacagctggttcgtgaacggcaccttccagcagagcacccaggagctgttcatccccaacatcaccgtgaacaacagcggcagctacacctgccaggcccacaacagcgacaccggcctgaaccgcaccaccgtgaccaccatcaccgtgtacgccgagccccccaagcccttcatcaccagcaacaacagcaaccccgtggaggacgaggacgccgtggccctgacctgcgagcccgagatccagaacaccacctacctgtggtgggtgaacaaccagagcctgcccgtgagcccccgcctgcagctgagcaacgacaaccgcaccctgaccctgctgagcgtgacccgcaacgacgtgggcccctacgagtgcggcatccagaacgagctgagcgtggaccacagcgaccccgtgatcctgaacgtgctgtacggccccgacgaccccaccatcagccccagctacacctactaccgccccggcgtgaacctgagcctgagctgccacgccgccagcaacccccccgcccagtacagctggctgatcgacggcaacatccagcagcacacccaggagctgttcatcagcaacatcaccgagaagaacagcggcctgtacacctgccaggccaacaacagcgccagcggccacagccgcaccaccgtgaagaccatcaccgtgagcgccgagctgcccaagcccagcatcagcagcaacaacagcaagcccgtggaggacaaggacgccgtggccttcacctgcgagcccgaggcccagaacaccacctacctgtggtgggtgaacggccagagcctgcccgtgagcccccgcctgcagctgagcaacggcaaccgcaccctgaccctgttcaacgtgacccgcaacgacgcccgcgcctacgtgtgcggcatccagaacagcgtgagcgccaaccgcagcgaccccgtgaccctggacgtgctgtacggccccgacacccccatcatcagcccccccgacagcagctacctgagcggcgccaacctgaacctgagctgccacagcgccagcaaccccagcccccagtacagctggcgcatcaacggcatcccccagcagcacacccaggtgctgttcatcgccaagatcacccccaacaacaacggcacctacgcctgcttcgtgagcaacctggccaccggccgcaacaacagcatcgtgaagagcatcaccgtgagcgccagcggcaccagccccggcctgagcgccggcgccaccgtgggcatcatgatcggcgtgctggtgggcgtggccctgatctga

SEQ ID NO: 2 - protein, 702 aa, Homo Sapien
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 3 - DNA, 2109 bp, Homo Sapien
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 4 - protein, 15 aa, Artificial Sequence
  peptide
TYYRPGVNLSLSCHA

SEQ ID NO: 5 - protein, 15 aa, Artificial Sequence
  peptide
NTTYLWWVNGQSLPV

SEQ ID NO: 6 - protein, 15 aa, Artificial Sequence
  peptide
YVCGIQNSVSANRSD

SEQ ID NO: 7 - protein, 15 aa, Artificial Sequence
  peptide
SASNPSPQYSWRING

SEQ ID NO: 8 - protein, 15 aa, Artificial Sequence
  peptide
VILNVLYGPDAPTIS

SEQ ID NO: 9 - protein, 15 aa, Artificial Sequence
  peptide
GPYECGIQNELSVDH

SEQ ID NO: 10 - protein, 15 aa, Artificial Sequence
  peptide
SPSYTYYRPGVNLSL

SEQ ID NO: 11 - protein, 15 aa, Artificial Sequence
  peptide
PSPQYSWRINGIPQQ

SEQ ID NO: 12 - protein, 15 aa, Artificial Sequence
  peptide
NNSIVKSITVSASGT

SEQ ID NO: 13 - DNA, 9 bp, Artificial Sequence
  Kozak Sequence
gccgccacc

SEQ ID NO: 14 - protein, 9 aa, Artificial Sequence
  T-cell epitope
CGIQNSVSA

SEQ ID NO: 15 - DNA, 2034 bp, Artificial Sequence
  hCEA- AD
atggagagccccagcgcccccccccaccgctggtgcatcccctggcagcgcctgctgctgaccgccagcctgctgaccttctggaacccccccaccaccgccaagctgaccatcgagagcacccccttcaacgtggccgagggcaaggaggtgctgctgctggtgcacaacctgccccagcacctgttcggctacagctggtacaagggcgagcgcgtggacggcaaccgccagatcatcggctacgtgatcggcacccagcaggccacccccggccccgcctacagcggccgcgagatcatctaccccaacgccagcctgctgatccagaacatcatccagaacgacaccggcttctacaccctgcacgtgatcaagagcgacctggtgaacgaggaggccaccggccagttccgcgtgtaccccgagctgcccaagcccagcatcagcagcaacaacagcaagcccgtggaggacaaggacgccgtggccttcacctgcgagcccgagacccaggacgccacctacctgtggtgggtgaacaaccagagcctgcccgtgagcccccgcctgcagctgagcaacggcaaccgcaccctgaccctgttcaacgtgacccgcaacgacaccgccagctacaagtgcgagacccagaaccccgtgagcgcccgccgcagcgacagcgtgatcctgaacgtgctgtacggccccgacgcccccaccatcagccccctgaacaccagctaccgcagcggcgagaacctgaacctgagctgccacgccgccagcaacccccccgcccagtacagctggttcgtgaacggcaccttccagcagagcacccaggagctgttcatccccaacatcaccgtgaacaacagcggcagctacacctgccaggcccacaacagcgacaccggcctgaaccgcaccaccgtgaccaccatcaccgtgtacgccgagccccccaagcccttcatcaccagcaacaacagcaaccccgtggaggacgaggacgccgtggccctgacctgcgagcccgagatccagaacaccacctacctgtggtgggtgaacaaccagagcctgcccgtgagcccccgcctgcagctgagcaacgacaaccgcaccctgaccctgctgagcgtgacccgcaacgacgtgggcccctacgagtgcggcatccagaacgagctgagcgtggaccacagcgaccccgtgatcctgaacgtgctgtacggccccgacgaccccaccatcagccccagctacacctactaccgccccggcgtgaacctgagcctgagctgccacgccgccagcaacccccccgcccagtacagctggctgatcgacggcaacatccagcagcacacccaggagctgttcatcagcaacatcaccgagaagaacagcggcctgtacacctgccaggccaacaacagcgccagcggccacagccgcaccaccgtgaagaccatcaccgtgagcgccgagctgcccaagcccagcatcagcagcaacaacagcaagcccgtggaggacaaggacgccgtggccttcacctgcgagcccgaggcccagaacaccacctacctgtggtgggtgaacggccagagcctgcccgtgagcccccgcctgcagctgagcaacggcaaccgcaccctgaccctgttcaacgtgacccgcaacgacgcccgcgcctacgtgtgcggcatccagaacagcgtgagcgccaaccgcagcgaccccgtgaccctggacgtgctgtacggccccgacacccccatcatcagcccccccgacagcagctacctgagcggcgccaacctgaacctgagctgccacagcgccagcaaccccagcccccagtacagctggcgcatcaacggcatcccccagcagcacacccaggtgctgttcatcgccaagatcacccccaacaacaacggcacctacgcctgcttcgtgagcaacctggccaccggccgcaacaacagcatcgtgaagagcatcaccgtgagcgccagcggc

SEQ ID NO: 16 - protein, 678 aa, Artificial Sequence
  hCEA- AD
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASG